(19) United States
(12) Patent Application Publication
(10) Pub. No.: US 2005/0084531 A1
(43) Pub. Date: Apr. 21, 2005

(54) Tablet with aqueous-based sustained release coating

(76) Inventors: Jatin Desai, Plainsboro, NJ (US); Roger Stanko, South Plainfield, NJ (US); Ronald W. Miller, Langhorne, PA (US); Richard Y. Ho, Murray Hill, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/965,034

(22) Filed: Oct. 14, 2004

Related U.S. Application Data
(60) Provisional application No. 60/511,830, filed on Oct. 16, 2003.

(51) Int. Cl. A61K 9/24; A61K 9/32; A61K 33/14

(52) U.S. Cl. 424/471; 424/679

(57) ABSTRACT

A tablet core containing a water-soluble, preferably highly water-soluble, active ingredient is coated for sustained release with an aqueous-based coating of an ethyl acrylate-methyl methacrylate copolymer. The amount of copolymer applied, on a dry basis, being about 0.5% to about 2% by weight, based on the total weight of the coated tablet. The coated tablet is dried for not more than about 30 minutes, preferably for about 10 to about 15 minutes, at about 50° C. Notwithstanding the greatly shortened drying time and/or low percentage of copolymner applied, the coated tablet surprisingly exhibits a substantially stable dissolution profile. Tablets containing potassium chloride and coated in accordance with the invention surprisingly exhibit a dissolution profile comparable to that afforded by potassium chloride tablets coated for sustained release with an organic solvent-based coating.

### CROSS REFERENCE TO RELATED APPLICATIONS

This application claims the benefit of U.S. Provisional Application No. 60/511,830, filed Oct. 16, 2003, incorporated herein by reference in its entirety.

### FIELD OF THE INVENTION

The invention relates generally to aqueous-based sustained release tablet coatings for highly water-soluble pharmaceutical active ingredient(s) that employ an amount of polymer substantially below that previously employed in the art while affording release rates comparable to those of a like tablet containing the highly water-soluble pharmaceutical active ingredient(s) but having an organic solvent-based sustained released coating.

Advantageously, tablets that contain highly water-soluble active ingredient(s) that are coated in accordance with the present invention utilize a drying step, at about 50° C., of only about 30 minutes, preferably 15 minutes. Furthermore, despite the use of a drying step that is less than taught as necessary by the prior art, tablets coated for sustained release in accordance with the present invention, surprisingly exhibit a substantially stable release profile, as hereinafter defined.

### BACKGROUND OF THE INVENTION

Because of environmental concerns and, in many instances, the need to comply with environmental laws and regulations, manufacturers of pharmaceuticals seek alternatives to coating tablets with volatile organic solvent-based coating systems. Aqueous-based coating systems are an alternative. However, when an FDA approved volatile organic solvent-based coated drug is involved, the replacement aqueous-based coating must provide drug release equivalent to the filed FDA approved volatile organic solvent-based coated drug.

Reducing the emission of volatile organic compounds by switching from a solvent-based coating to an aqueous-based coating without changing the release profile of a tablet containing a water-soluble drug, particularly a highly water-soluble drug, such as potassium chloride or metformin hydrochloride, is not a simple task.

For example, based on the coating or polymer coating manufacturer's literature and in accordance with the teachings of current technology, aqueous-based Eudragit® dispersion coatings are applied on granules, beads, crystals or tablets so that typically from 5 to 40%, based on the weight of the core, of dry Eudragit® polymer is applied. According to the coating manufacturers' literature and what is known in the art, unless the coated granules, beads, or crystals are subjected to long term curing, the release profile of the active ingredient will change during storage, such that the release profile becomes slower. Consequently, tablets coated with prior art sustained release coating compositions are typically cured for 8 to 24 hours at a temperature of 40 to 60° C.

At the outset, it should be noted that, as used herein, a “highly water-soluble active ingredient” is an active ingredient that has a dose/solubility volume greater than or equal to 5 mg/ml. Further, the term drug and active ingredient are used interchangeably herein and are synonymous. Additionally, unless otherwise indicated, as used herein, percentage is percent by weight based on total weight.

Potassium chloride, a highly water-soluble compound, is marketed pursuant to an approved New Drug Application as KLOTRIX® tablets. Such tablets are coated for sustained release using a volatile organic solvent-based coating system. As presently marketed, coated potassium chloride tablets are printed on the barrier coating. Engraved tablets are not employed for fear that the barrier coating will not be uniform and the release rate will be adversely affected. Potassium chloride tablet cores are disclosed in U.S. Pat. No. 4,140,756. However, the cores are disclosed in conjunction with an organic solvent-based coating and not with an aqueous-based coating system.

U.S. Pat. No. 4,140,756 discloses a film-coated matrix core tablet for the continuous controlled release of a water-soluble medicament, such as potassium chloride, or a dietary supplement, over a prolonged dissolution period of at least about five hours. This patent describes a water insoluble wax-like matrix core containing the water-soluble medicament that is coated with a permeable erosion resistant polymeric film. As indicated above, the preferred film coat does not employ aqueous-based coatings. Instead volatile organic compounds, such as alcohol and methylene chloride, are used in the process which requires expensive stripping and recovery steps in order to keep the resulting volatile organic compound (VOC) output levels within EPA standards.

PCT Application WO 99/42087 relates to a controlled release composition, preferably in the form of tablets or hard gelatin capsules and having 500 to 1000 mg potassium chloride per dosage unit. At least 70% by weight of the potassium chloride is in the form of coated or partially uncoated pellets. The tablets or hard gelatin capsules comprise pellets containing at least 70% by weight potassium chloride, 10-25% by weight microcrystalline cellulose, 0.1-0.5% by weight anti-adhesion agent and 0.1-5% by weight hydrophobic agent. The coating layer that is applied to the pellets comprises 3-10% by weight of ethyl acrylate/methyl methacrylate copolymer and/or ammonium methacrylate copolymer, hydrophobic agent, 5-35% by weight of a lower alkanol, talc and, optionally, a dye. Potassium chloride particles and further auxiliary agents may be applied onto the coating layer. Accordingly, the finished tablet is not coated. Rather, potassium chloride pellets are first coated with polymers by the use of an organic solvent (a lower alkanol), and then are further processed and compressed into a tablet. In addition, WO 99/42087 teaches away from the use of an aqueous-based coating system by noting that the use of water in the formation of the composition is accompanied by an “unfavorable phenomenon” (see page 16, first paragraph).

U.S. Pat. No. 5,651,984 discloses a pharmaceutical dosage form prepared from a multiplicity of coated potassium chloride crystals coated with a first layer of ethyl cellulose and a second layer of a hydrophilic coating polymer, preferably hydroxypropyl cellulose. The resultant microcapsules can be compressed into controlled release tablets. Accordingly, the finished tablet is not coated. Rather, potassium chloride microcrystals are first coated with polymers by the use of an organic solvent (cyclohexane), and then further processed and compressed into a tablet.

U.S. Pat. No. 5,500,227 relates to a controlled release tablet having a core containing an insoluble therapeutically active agent. The core provides rapid release of the active upon exposure to aqueous solutions. The tablet core is coated with a controlled release coating permitting sustained release of the active when the tablet is exposed to aqueous solutions. In a preferred embodiment, the film coating is obtained by use of an aqueous dispersion of a hydrophobic polymer such as ethyl cellulose, a polymer or copolymer of acrylates or methacrylates or a mixture thereof. The coating is applied so that it increases tablet weight from about 3 to 20% (see col. 3, lines 38-43). It was discovered that controlled release formulations of insoluble drugs can be prepared with batch-to-batch and scale-up reproducibility of in-vitro dissolution by over coating immediate release tablet cores, containing the insoluble drug, with a controlled release film coating. The invention of U.S. Pat. No. 5,500,227 is directed to drugs having low solubility, not to highly water-soluble active ingredients such as potassium chloride.

U.S. Pat. No. 4,784,858 discloses a controlled release tablet comprising (I) a core that contains at least one water-soluble active dispersed in a water-insoluble, non-digestible polymeric excipient and a water-insoluble polymeric substance swellable under the influence of water and (II) a core essentially of an elastic, water-insoluble and semi-permeable diffusion film of a polymer. The tablet has a release pattern for the active in a programmed rate of approximately zero order. The elastic, water-insoluble and semi-permeable diffusion film of a polymer essentially consists of a homo-or copolymer of lower alkyl acrylates and/or lower alkyl methacrylates, alone or mixed with a latex (aqueous suspension) of ethyl cellulose. In Example 1, a core is prepared containing actives by granulating the actives with aqueous polyvinylpyrrolidone and passing same through a sieve, then spraying the sieved granules with a 30% aqueous dispersion of 70:30 copolymer of ethyl acrylate and methyl methacrylate (Eudragit®-E30D). The dried coated granules are mixed with microcrystalline cellulose, magnesium stearate and colloidal silicon dioxide, sieved, mixed and then compressed into tablets weighing 1.097 g. The tablets are coated with 0.187 kg Eudragit®-E30D, 0.047 kg talcum, 0.004 kg polysorbate 80, 1.5 g indigo-tin lake and 0.75 g titanium dioxide in 500 g of water. The amount of film coating sprayed on is 31 mg (dry weight). Thus each coated tablet weighed 1.128 g (1.097 g+0.031 g). In other words, the coating represents 2.7% of the coated tablet weight, on a dry basis. The coating contains 187 g Eudragit®-E30D in 286.25 g of total coating solids. Thus 65.33% of the coating (on a dry basis) is Eudragit®. Since each coated tablet contains 2.7% coating (on a dry basis) and 65.32% of the coating is Eudragit®, the coated tablet has (65.33%×2.7%) or 1.76% Eudragit® in the coating layer, a high level of Eudragit® in the coating. Moreover, the tablets employ two coatings. The granules are coated, the coated granules are tableted, and then the tablets are coated, requiring a high total amount of Eudragit®.

European Patent application 0171457B1 discloses a composition for controlled discharge of an active and a method for preparing same. A prill seed is disclosed containing a water-soluble active surrounded by a semi-permeable membrane containing a particulate water-soluble pore forming material which dissolves to form pores in the semi-permeable membrane, which is permeable to water but not to the active, enabling the active to be dissolved and an osmotic pressure gradient to be created between the solution and the aqueous environment. The prill seed formulation is coated with a coating mix that is organic solvent-based (see col. 7, lines 12-21). Thus, this process employs a volatile organic solvent-based system and the coating does not cover the entire tablet, merely the prill seeds.

European Patent application 0211991 discloses a sustained release coated drug-containing tablet. The coating is made up of a polymer insoluble in water and gastro-intestinal fluids, and a highly water-soluble pore creating substance randomly distributed in the polymer. The pore creating substance is substantially pharmaceutically inactive in the amount used and consists of particles essentially insoluble in the solvent used to coat the tablet. The polymer is a terpolymer of vinyl chloride, vinyl acetate and vinyl alcohol. The pore creating substance is present in an amount of 1-20 parts for each 1-10 parts of terpolymer. The coating is prepared by dissolving the terpolymer in a solvent such as acetone, methylene chloride, methyl ethyl ketone or a mixture of acetone and ethanol, acetone and methylene chloride, or the like (see page 3, lines 46-48). Thus, it is clear that the coating is volatile organic solvent-based and not aqueous-based.

There remains a need for an environmentally sound aqueous-based tablet coating system that can be employed to coat a tablet containing a water-soluble drug, particularly, a highly water-soluble drug, whereby the release rate of the drug is substantially the same as the release rate of the drug from the volatile organic solvent-based coated tablet.

### SUMMARY OF THE INVENTION

It is an object of the present invention to provide an aqueous-based sustained release coating for tablets containing water-soluble, preferably highly water-soluble, active ingredients.

It is another object of the invention to provide an aqueous-based sustained release coating for tablets containing water-soluble, preferably highly water-soluble, active ingredients, wherein the coating utilizes a greatly short drying time yet exhibits a substantially stable release profile (as hereinafter defined).

It is yet another object of the invention to provide an aqueous-based sustained release coating for tablets containing water-soluble, preferably highly water-soluble, active ingredients, particularly potassium chloride, wherein the coating utilizes a short drying time yet exhibits a substantially stable release profile that substantially mirrors the release profile of a like tablet that contains such active ingredient but is coated for sustained release with an organic solvent-based coating.

It is a further object of the invention to reduce processing time and energy costs by decreasing the time needed to produce tablets coated with sustained release coatings, thereby allowing substantial manufacturing cost savings to be realized.

It is a still further object of the invention to reduce VOCs by providing an aqueous-based sustained release coating for tablets containing water-soluble, preferably highly water-soluble, active ingredients, as an alternative to organic solvent-based sustained release coatings.

### DETAILED DESCRIPTION OF THE INVENTION

The present invention is directed to an aqueous-based tablet coating system that can be employed to coat a tablet containing a water-soluble drug, particularly, a highly water-soluble drug, and the release rate of the drug is substantially the same as the release rate of the drug from the volatile organic solvent-based coated tablet. More importantly, and surprisingly, the aqueous-based coating system of the present invention is able to accomplish this while employing an amount of polymer greatly below amounts taught as necessary by prior art.

Still further, the coating compositions of the present invention can surprisingly be used to coat embossed tablets. There is no need to drill a hole in the coating (mechanically or laser drilled) or to use one or more additional osmotic agent(s) or to subject the coated tablets to a curing operation, to ensure operability.

Thus, another surprising advantage of tablets coated with the aqueous-based coating composition of this invention is that they do not utilize long term curing.

A further surprising advantage is the fact that the coated tablets of the present invention can be stored under accelerated stability conditions for up to six months and the water-soluble active ingredient contained therein will still have an acceptable dissolution profile.

Tablets that contain a highly water-soluble active ingredient and that are coated with a coating composition of the present invention do not need to be cured for long periods of time and/or at high temperatures. Rather, the tablets of the present invention can be coated with a drying step. Optimally, the drying step is carried out for about 10 to about 15 minutes at 50° C. A short drying time is all that is used, as opposed to long term curing, because, surprisingly, the release profile of the highly water-soluble active ingredient contained in the sustained release coated tablet produced in accordance with the present invention does not substantially change during long term storage at 40° C. and 75% relative humidity.

Although the present invention is described with reference to tablets containing potassium chloride as the principal active ingredient, it should be appreciated that the invention is also applicable to delivery of other water-soluble, preferably highly water-soluble, active ingredients. For example, water-soluble compounds, or pharmaceutically acceptable salts thereof, typically used for therapies of diabetes, hypertension, psychiatric disorders, electrolyte imbalance, etc. Active ingredients such as potassium chloride, metformin hydrochloride (Glucophage®), omapatrilat, captopril, hydrochlorthiazide, and the like, could be designed for 8 hour, 12 hour, or once-a-day dosage.

Prior art coatings containing Eudragit® that are applied for adequate sustained release applications generally range from 4.0% to 12.0% by weight, typically 6.0% to 10.0%, based on the weight of the core substrate to which the coating is applied. In contrast thereto, the present invention provides a tablet containing a high dose of a water-soluble active ingredient, preferably a highly water-soluble active ingredient, more preferably, potassium chloride or metformin hydrochloride, and most preferably potassium chloride. The tablet is coated with an aqueous-based coating comprised of an ethyl acrylate-methyl methacrylate copolymer, for example, Eudragit® NE30D (Rohm America) or Kollicoat® EMM 30D (BASF), said coating ranging from about 2% to about 3.5%, preferably from about 2.25% to about 2.75%, most preferably which the coating is applied. Surprisingly, in contrast to what is expected in the art, the coated compositions of the present invention containing such low percentages of copolymer applied provide a satisfactory sustained release profile that remains substantially stable over time.

Preferred coatings of the present invention comprise the following components:

| | Preferred range | More preferred |
|---|---|---|
| | (% total dry | range (% total dry |
| Component | weight) | weight) |
| Ethyl acrylate - methyl methacrylate | 0.63-1.17 | 0.800-0.988 |
| copolymer (e.g. Eudragit ® NE30D) | | |
| Polyethylene glycol (“PEG”) | 0.08-0.16 | 0.107-0.132 |
| Precipitated silica (e.g. Syloid Silicon | 0.05-0.88 | 0.060-0.073 |
| Dioxide 244 FP) | | |
| Lactose (e.g. Lactose DT anhydrous, | 0.31-0.58 | 0.390-0.489 |
| NF) | | |
| Talc (e.g. Soft talc, USP) | 0.62-1.16 | 0.790-0.976 |
| Antifoam agent (e.g. Simethicone | 0.01-0.03 | 0.018-0.024 |
| emulsion (30%)) | | |

It should be noted that “substantially stable dissolution profile” as used herein and in the claims that follow, means the dissolution of coated tablets that have been stored for 26 weeks at 40° C. and 75% relative humidity in open dishes that do not deviate by more than 5 and preferably 2% from the dissolution profile determined for the coated tablets at substantially the time of their production, preferably within two weeks.

The aqueous-based coating of the present invention is comprised of an ethyl acrylate-methyl methacrylate copolymer, for example, Eudragit® NE30D or Kollicoat® EMM 30D (methacrylic acid copolymer Type C), as the coating agent. Eudragit® NE 30D and Kollicoat® EMM 30D are ethyl acrylate-methyl methacrylate copolymer dispersions, 30%. They have been assigned the EP official name of Polyacrylate dispersion 30%. Eudragit® NE 30D has been referred to as Poly (EA-MMA) 2:1 (see “Chemistry and Application Properties of Polymethacrylate Coating Systems”, Klaus, O. R. Lehman, reprinted from “Aqueous Polymeric Coatings for Pharmaceutical Dosage Forms”, 2^{nd} Edition, Revised and Expanded, Edited by James McGinity, 1996).

Industry standard Eudragit®-based sustained release coatings are disclosed as being cured for from 6 to 18 hours at 40 to 60° C. Typically, they are cured for from 10 to 12 hours at 60° C. It is well known in the art that if they are not subjected to this protracted curing operation the release rate of the coated tablet will change on storage. In contradistinction thereto, the aqueous-based coating of the present invention merely uses drying for not more than 30 minutes, preferably from about 10 to about 15 minutes, at about 50° C. The processing time saved and the energy cost savings realized, due to the greatly shortened drying step, translates into substantial manufacturing cost savings.

Organic solvent-based coated KLOTRIX® tablets (2.5% coating) have the following dissolution profile:

| | |
|---|---|
| 1 hour | 8-25% |
| 2 hours | 27-51% |
| 3 hours | 45-75% |
| 5 hours | at least 66% |
| 7 hours | at least 90%. |

It should be appreciated that the examples that follow serve only to exemplify the various aspects of carrying out the present invention and are not intended to limit the invention in any way.

### EXAMPLES

### Example 1

In accordance with the present invention, tablet cores containing 750 mg potassium chloride, povidone (USP), FD&C Yellow #6 Lake, stearic acid powder, magnesium stearate and water, wherein the total weight of the tablet core was 916.80 mg, were prepared by methods known to one of ordinary skill in the art and then coated using an aqueous-based coating composition, wherein the total weight of the coated tablet was only 939.72 mg.

| | | Dry Basis | Dry Basis |
|---|---|---|---|
| | | % of | % of |
| Ingredient | Mg/tablet | each part | coated tablet |
| Coating Suspension | | | |
| Eudragit ® NE 30D (30% solids) | 8.44564 | 36.848 | 0.899 |
| PEG E1450, NF | 1.12531 | 4.910 | 0.120 |
| Syloid silicon dioxide 244FP | 0.62517 | 2.728 | 0.067 |
| Lactose DT anhydrous, NF | 4.17161 | 18.201 | 0.444 |
| Soft talc, USP | 8.34323 | 36.402 | 0.888 |
| Simethicone emulsion (30%) | 0.19324 | 0.843 | 0.021 |
| (Dow Corning, #7-9245) | | | |
| FD&C Yellow #6 Lake | 0.01591 | 0.069 | 0.002 |
| Purified water (q.s. to 20.164% | Q.S. | | |
| solids suspension) | | | |
| Total: | 22.920 | 100.000 | 2.439* |

*The tablet cores containing 750 mg potassium chloride, povidone (USP), FD&C Yellow #6 Lake, stearic acid powder, magnesium stearate and purified water account for the remaining percentage (97.561%) of the tablet.

A. Preparation of the Coating Suspension

Charge the water into a suitable solution preparation tank equipped with an agitator. While mixing, sequentially charge the simethicone emulsion, polyethylene glycol E1450, lactose, Syloid, and soft talc into the tank and mix to form a dispersion. Add the Eudragit® NE 30D thereto, under slow agitation, to form the coating suspension.

B. Procedure for Tablet Coating

125-150 kg of potassium chloride tablet cores are charged into a 48″ perforated coating pan (preferably a 48″ Accela-Cota coating pan). Preheat the tablets to 30-35° C. exhaust temperature using an inlet air temperature of 45-60° C. with jogging. Film coat the tablets with the coating suspension produced in step A above using the following parameters:

(i) Air flow of 1800 to 2100 cfm

(ii) Pan speed of 3 to 7 rpm

(iii) Atomization pressure of 40 to 55 psi

(iv) Suspension spray rate of 280 to 450 g/min (keep suspension agitated to prevent solids from settling)

(v) Inlet air temperature of 45 to 60° C.

(vi) Exhaust air temperature of 28 to 35° C.

(vii) Spray distance of 8″ to 12″

(viii) Inlet dew point of 30 to 55° F.

### Example 2

In accordance with the present invention, compressed tablet cores containing 500 mg metformin hydrochloride, povidone (USP), magnesium stearate and water (USP), wherein the total weight of the tablet core was 529.99 mg, were prepared by methods known to one of ordinary skill in the art and then coated using an aqueous-based coating composition. Such tablets had acceptable dissolution profiles and demonstrate that the coatings of the present invention are suitable for modified release with multiple core tablet formulations containing highly water-soluble actives.

| Ingredient | Suspension/Grams | % (w/w) |
|---|---|---|
| Coating Suspension | | |
| Eudragit ® NE 30D (30% solids) | 1730.00 | 24.767 |
| PEG E1450, NF | 69.30 | 0.990 |
| Syloid silicon dioxide 244FP | 38.50 | 0.550 |
| Lactose DT anhydrous, NF | 256.90 | 3.670 |
| Soft talc, USP | 513.80 | 7.340 |
| Simethicone emulsion (30%) | 11.90 | 0.170 |
| (Dow Corning, #7-9245) | | |
| FD&C Yellow #6 Lake | 0.98 | 0.014 |
| Cold deionized water | 4375.00 | 62.499 |
| Total: | 6996.38 | 100.00 |

Compressed tablet cores containing 500 mg metformin hydrochloride, povidone (USP), magnesium stearate and water (USP) were preheated to 30 to 35° C. exhaust temperature in an Accela-Cota pan and then coated with the coating composition set forth above until 2.5% and 4% of theoretical solids had been applied. The tablets were dried for 15 minutes at 50° C. inlet temperature with continuous rotation at the lowest pan speed. The dissolution profiles of the tablets coated with coating suspension for a 2.5% theoretical weight gain and the tablets coated with coating suspension for a 4% theoretical weight gain were determined. The release (or dissolution) rates were determined by liquid chromatography (LC) as follows:

% of metformin HCl dissolved

| | |
|---|---|
| | 2.5% |
| 4% Coating | Coating |

| Hour | * | ** | * | ** |
|---|---|---|---|---|
| 1 | 21.4 | 18.5 | 28.8 | 30.4 |
| 2 | 41.2 | 36.7 | 53.8 | 59.6 |
| 3 | 61.4 | 53.0 | 74.8 | 81.4 |
| 5 | 89.6 | 81.5 | 101.0 | 97.8 |
| 7 | 98.7 | 97.6 | 101.6 | 98.6 |

* - based on an average of three tablets
** - based on an average of six tablets

The contents of all patents, patent applications, published articles, books, reference manuals and abstracts cited herein are hereby incorporated by reference in their entirety to more fully describe the state of the art to which the invention pertains.

As various changes can be made in the above-described subject matter without departing from the scope and spirit of the invention, it is intended that all subject matter contained in the above description, or defined in the appended claims, be interpreted as descriptive and illustrative, and not in a limiting sense. Modifications and variations of the present invention are possible in light of the above teachings. It is therefore to be understood that within the scope of the appended claims, the invention may be practiced otherwise than as specifically described.

1. A tablet having a tablet core containing a highly water-soluble active ingredient and an aqueous-based coating on said core adapted for sustained release of said active ingredient comprising an aqueous-based coating on the core that when dried has about 0.5% to about 1.17%, by weight based on the total weight of the coated tablet, of an ethyl acrylate-methyl methacrylate copolymer and the coating is dried for not more than about 30 minutes at about 50° C., whereby said tablet provides sustained release of said active ingredient and said active ingredient has a dissolution profile that is substantially stable.

2. The tablet as claimed in claim 1, wherein the coating contains about 0.800% to about 0.988% of copolymer.

3. The tablet as claimed in claim 1, wherein the coating is dried from about 10 to about 15 minutes.

4. The tablet as claimed in claim 1, wherein the active ingredient is potassium chloride, metformin hydrochloride, omapatrilat, captopril, hydrochlorthiazide or a pharmaceutically acceptable salt thereof.

5. The tablet as claimed in claim 1, wherein the active ingredient is potassium chloride.

6. The tablet as claimed in claim 1, wherein the active ingredient is metformin hydrochloride.

7. The tablet as claimed in claim 1, wherein the coating is dried from about 10 to about 15 minutes, the active ingredient is potassium chloride, and the coated potassium chloride containing tablet has the following release profile:

| | |
|---|---|
| 1 hour | 8-25% |
| 2 hours | 27-51% |
| 3 hours | 45-75% |
| 5 hours | at least 66% |
| 7 hours | at least 90%. |

8. The tablet as claimed in claim 1, wherein the coating contains an ethyl acrylate-methyl methacrylate copolymer, polyethylene glycol, precipitated silica, lactose, talc, and an antifoam agent.

9. The tablet as claimed in claim 1, wherein the coating contains, by weight based on the weight of the coated tablet:
about 0.5% to about 1.17% of an ethyl acrylate-methyl methacrylate copolymer;
about 0.08% to about 0.16% of polyethylene glycol;
about 0.05% to about 0.88% of precipitated silica;
about 0.31% to about 0.58% of lactose;
about 0.62% to about 1.16% of talc; and
about 0.01% to about 0.03% of an antifoam agent.

10. The tablet as claimed in claim 1, wherein the coating contains, by weight based on the weight of the coated tablet:
about 0.63% to about 1.17% Eudragit® NE 30D;
about 0.08% to about-0.16% Polyethylene glycol E1450;
about 0.05% to about 0.88% Syloid silicon dioxide 244FP;
about 0.31% to about 0.58% Lactose DT anhydrous, NF;
about 0.62% to about 1.16% Soft talc, USP; and
about 0.01% to about 0.03% Simethicone emulsion (30%) (Dow Corning, #7-9245).

11. The tablet as claimed in claim 10, wherein the coating contains, by weight based on the weight of the coated tablet:
about 0.90% Eudragit® NE 30D.
about 0.12% Polyethylene glycol E1450;
about 0.07% Syloid silicon dioxide 244FP;
about 0.44% Lactose DT anhydrous, NF;
about 0.89% Soft talc, USP; and
about 0.02% Simethicone emulsion (30%) (Dow Corning, #7-9245).
